(19) United States
(12) Patent Application Publication
(10) Pub. No.: US 2013/0017182 A1
(43) Pub. Date: Jan. 17, 2013

(54) MULTI-CRITERIA OPTIMIZED DIETARY SUPPLEMENT FORMULATIONS

(71) Applicant: Natalia Lukina, Valley Cottage, NY (US)

(72) Inventor: Natalia Lukina, Valley Cottage, NY (US)

(21) Appl. No.: 13/545,856

(22) Filed: Jul. 10, 2012

Related U.S. Application Data
(60) Provisional application No. 61/506,630, filed on Jul. 11, 2011.

(51) Int. Cl. A61K 38/43; A61K 36/45; A61P 3/02; A23G 3/36; A23L 1/30; A23L 1/304; A23G 4/06; A61K 31/122; A23L 1/302

(52) U.S. Cl. 424/94.1; 514/690; 424/732; 426/72; 426/648; 426/74; 426/3; 426/573; 426/96

(57) ABSTRACT

Dietary micronutrient supplement formulations for specific ages, gender, special requirements and health conditions comprising, vitamins, minerals, fish and plant oils, amino acids, enzymes, phytochemicals, herb and fruit extracts and other natural compounds grouped into morning, mid-day and evening formulas based on their synergism and antagonism with each other, their interactions with ingredients in the food consumed during each meal and their bioavailability. The dietary micronutrient formulations are optimized to meet the Recommended Daily Allowances (RDA) and Adequate Intake (AI) standards for each segment of the population with larger amounts of nutrients used in condition-specific formulas.

### CROSS REFERENCE TO RELATED APPLICATION

This application claims priority to U.S. Provisional Patent Application Ser. No. 61/506,630 filed Jul. 11, 2011, the subject matter of which is incorporated herein in its entirety.

### FIELD OF THE INVENTION

The present invention is generally related to nutritional supplements. More particularly, the invention is related to micronutrient supplements that are formulated to provide optimal intake and absorption by the human body to carry out its daily functions, in health and disease.

### BACKGROUND OF THE INVENTION

Micronutrients are nutrients required only in minute amounts by the human body, but nevertheless play a critical role in the normal growth and development of the body. Deficiencies in micronutrients can lead to a breakdown in numerous bodily functions and result in a plethora of mild to severe diseases. Since the human body is not capable of synthesizing most of the essential micronutrients, the only way to obtain them is through dietary food sources or through supplementation.

The principal micronutrients fall into two categories-vitamins and minerals. Vitamins are essential micronutrients that the body is not capable of synthesizing in sufficient quantities for its growth and maintenance and have to be derived from dietary food sources. However, most vitamins are present only in minute quantities in the foods that we ingest and their bioavailability depends on the food sources. There are thirteen essential vitamins of which four, A, D, E, and K are fat soluble and nine, B1, B2, B3, B6, B12, pantothenic acid, biotin, folic acid, and C are water soluble. The fat soluble vitamins are capable of being retained in the body while the water soluble vitamins are excreted from the body. Therefore, it is critical to ensure adequate daily intake of the water soluble vitamins either through food or supplements.

Minerals which form the second category of micronutrients are inorganic in nature and can be broken down into two sub-categories: macrominerals such as calcium (Ca), phosphorous (P), sodium (Na), potassium (K), magnesium (Mg), and chloride (Cl) and trace minerals such as iron (Fe), zinc (Zn), iodine (Io), selenium (Se), copper (Cu), manganese (Mn), fluoride (fl), chromium (Cr) and molybdenum (Mo). Just as with the essential vitamins, these mineral micronutrients are essential for bodily functions and cannot be synthesized by the body. Therefore, it is necessary to have an adequate intake of these mineral micronutrients from food sources or through supplementation.

Besides vitamins and minerals, there are other micronutrients such as phytochemicals, amino acids and other natural compounds whose beneficial role in the human body have been documented by a number of clinical trials. Such compounds include CoQ-10, lutein, lycopene, astaxanthin, resveratol, flavonoids, 1-carnitine, inositol and others which are available through various food sources. However, the frequency of consumption of foods rich in these compounds and the bioavailability of these compounds from the food sources may be limited and therefore supplementation may be beneficial.

Herbal and fruit extracts have long been used to help maintain health. A number of herbs have been shown to have positive effects on various organs and systems of the body. For example, the positive effects of hawthorn on the heart and blood pressure, chamomile and ginger on digestive health, milk thistle on liver health, hops as a relaxing and anti-inflammatory agent, have all been scientifically documented. Among fruits, bilberry has been noted for its positive effect on eye functions, especially night-time visual acuity, and pomegranate for its antioxidant and anti-inflammatory properties. Not many in the general population consume these herbs and fruits to experience their beneficial effects and therefore, incorporation of standardized extracts of these or other generally safe and beneficial herbs and fruits into the daily multivitamin and mineral supplements routinely taken by many can facilitate their intake to provide the health benefits attributed to such herbs and fruits.

Omega-3 fish oil has been studied for many years and has many proven health benefits. The essential omega -3 fatty acids DHA and EPA have been shown to be anti-inflammatory and have beneficial effects on the heart. Since our water sources are getting more and more contaminated with heavy metals and other waste, consumption of fish from these water sources pose a particular challenge and concern for fish lovers. Since fish is the main source of natural omega-3 fatty acids in the diet, supplementation with high quality purified fish oil becomes essential in order to reap the health benefits of omega-3 fatty acids from a source other than through the consumption of fish.

In practice, most people do not consume a healthy and balanced diet that meets the Recommended Daily Allowances (RDA) for the essential micronutrients. In addition, under certain conditions such as smoking, alcohol consumption, stress, physical exertion, age, chronic disease and environmental exposure to the elements, the nutritional needs of the body are generally elevated due to inadequate intake and absorption of the essential micronutrients from food sources, requiring, vitamin, mineral and other micronutrient supplementation to keep the body in a healthy state.

While there are many daily vitamin-mineral formulas in the market that include vitamins, minerals, phytochemicals and herbs, most of them have significant nutritional imbalances. Many formulas in the market also fail to consider consumer's dietary choices, micronutrient interactions and often provide inadequate doses of some essential nutrients (often magnesium, calcium, copper, zinc, vitamin D) and mega-doses of other essential nutrients (vitamins B, C, E, A) without scientific validation of safety and efficacy of such doses during long-term use.

Most of the daily multivitamin and mineral formulations currently available in the market include combinations of micronutrients that are counterproductive and inhibit absorption of one another (such as for instance the combination of non-heme iron, the form of iron used in a vast majority of multivitamins, and calcium; iron and zinc; copper and zinc; manganese and magnesium, and especially manganese and a combination of magnesium and calcium; iron and manganese; flavonoids/polyphenolic compound and iron). Non-balanced doses and especially mega-doses of some vitamins or minerals can deplete other related nutrients, such as in the case of interaction between copper and zinc, certain B vitamins, magnesium and calcium, and others.

It is known that some micronutrients act synergistically and greatly affect absorption of one another. For example, ascorbic acid (Vitamin C) greatly enhances iron absorption when they are ingested together and the citrus bioflavonoids, in particular, rutin and quercetin, enhance vitamin C action; addition of oil significantly enhances bio-availability of the fat soluble vitamins, carotenoids and CoQ-10. Inclusion of synergistic ingredients is an important part of a balanced daily supplement; however, there are very few supplements in the market that have used that criteria and principle in their formulations.

It is also important to pay attention to the common food choices of the population. Many people drink coffee and tea in the morning and take supplements at or close to that time. Research has shown that coffee and tea reduce iron absorption by over 60% and possibly by as much as 80%. Another example is the consumption of meat or fish for dinner followed by a multivitamin supplement with Ca, Zn, and Mg, which will have an inhibitory action on the absorption of iron from the meal. When a multivitamin supplement with the fat soluble vitamins, A, D, E, and K is taken on an empty stomach or with a fruit or a rice cracker, absorption of its fat soluble components will be greatly impaired, since there was no fat consumed together with the supplement. Currently, multivitamin products fail to recognize such interactions and to provide this information to consumers.

When micronutrients that have antagonistic or inhibitory actions on one another are included together in a supplement, they are of scant nutritional benefit to the body because they are either poorly absorbed or excessively excreted. With such a supplement, higher doses of the respective micronutrients are required to achieve a net positive beneficial effect on the body as compared to a supplement that contains synergistic nutrients.

Since a majority of the multivitamin supplements currently available in the market do not include all the essential micronutrients, there is a tendency for the consumer to combine the intake of the generic multivitamin tablets with other supplements that are specifically formulated as for instance, omega-3 fish oil capsules, calcium/magnesium tablets, antioxidant formula etc. Since some of these supplements may include a few of the same ingredients present in the generic multivitamin, there is a tendency to over consume a particular nutrient. For example, most daily multivitamins include vitamin A and D and do not include omega-3 fish oil. Omega-3 fish oil capsules on the other hand include vitamins A and D. Thus a consumer taking a daily multivitamin along with an Omeg-3 fish oil capsule will end up consuming excess amounts of vitamins and A and D beyond the daily requirements which can lead to toxicity since vitamins A and D are fat soluble vitamins not excreted by the body.

What is needed is a daily essential micronutrient supplement that is balanced and optimized according to nutrient interactions, time-of-day when it is used, medium of nutrient delivery, and bioavailability of nutrients. In addition, an effective micronutrient supplement needs to be balanced in terms of the nutrient requirements for age, gender and specific health conditions. The present invention provides such a micronutrient supplement.

### SUMMARY OF THE INVENTION

The present invention relates to micronutrient supplement formulations created with the goal of optimizing their intake and absorption by the human body under various conditions both in health and disease states. At the heart of the concept is the idea of combining the essential micronutrients into several distinct formulas that have the highest potential health benefit to the body without adverse side effects.

In an exemplary embodiment of the invention, the micronutrient supplement formulations have seven levels of optimization to increase nutrient bioavailability under a variety of conditions. In this embodiment of the invention, the 7-level optimization protocol is based on, 1) Time of day, 2) Meal content, 3) Medium of delivery, 4) Synergism of components, 5) Bio-availability of forms/compounds, 6) Age/gender, and 7) Special requirements and health conditions.

In the preferred exemplary embodiment of the invention, all the essential micronutrients are divided up and grouped together in a supplement formula according to their optimal effectiveness on the body during the time of the day, morning, mid-day, and evening, resulting in three supplement formulas to be taken during the specified times of the day or four formulas for special conditions.

In yet another preferred embodiment of the invention, consideration is given to the optimization of the absorption of the micronutrients by the body based on the type of meal and the contents of the meal so that the micronutrients in the supplement formula taken along with that meal are not inhibited from getting absorbed due to the potential negative interactions with ingredients in the meal.

In a related embodiment of the invention, the micronutrient supplement formulas are prescribed to be taken with the appropriate meals for effective absorption while others are formulated for ingestion on an empty stomach or in between meals.

In the exemplary embodiment of the invention, the micronutrients in the supplement formulas of the invention are grouped together based on the synergism for each other.

In yet another preferred embodiment of the invention, attention is given to the bioavailability of the micronutrients included in the supplement formulas so that they are in a form that is easily absorbed and retained by the body.

In the preferred embodiments of the present invention, age and gender are taken into account in the formulation of the micronutrient supplements and they are age specific based on the Recommended Dietary Allowances (RDA) and Adequate Intake (AI) norms for nutrients and for those nutrients that do not have established standards, based on the amounts that may be derived from a diet rich in fruits, vegetables and nutrient dense foods.

In a related preferred embodiment of the present invention, the special requirements and health conditions of the individual is taken into consideration in the formulation of the micronutrient supplements specifically intended for that segment of the population.

In this summary of the invention and in the specification in general, the various references to “exemplary embodiment,” “preferred embodiment,” “yet another embodiment,” “yet another preferred embodiment,” or “related embodiment” do not all necessarily refer to the same embodiment (s). Rather, these references in general mean that a particular embodiment is included in at least some embodiments, but not necessarily all embodiments of the invention. In general, the features and advantages of the embodiments of the present invention as described in this summary of the invention will be appreciated and will become obvious to one skilled in the art when viewed in conjunction with the detailed description of the invention accompanied by the tables and the appended claims.

### DETAILED DESCRIPTION OF THE INVENTION

The present invention provides micronutrient supplements that are formulated for optimal intake and absorption by the human body to meet the daily requirements for essential and beneficial micronutrients that are not met by dietary food sources. Unlike other nutritional supplements available in the market today which require the combining of multiple supplements to overcome the deficiencies for these nutrients from food sources, the micronutrient supplement formulations of the present invention have all the nutrients in the right combination available from a single source, tailored to meet the nutrient needs of different segments of the population.

Referring now to the micronutrient supplements of the present invention, each of the supplements are formulated to meet the daily requirements and physiological needs of a specific segment of the population. Examples of these population segments are 1) adults, 2) adult females, 3) adult males, 4) toddlers, 1-3, 5) children, 4-8, 6) juniors, 9-14, 7) pregnant women, 8) adults who need cardio/cholesterol support, 9) adults with iron deficiency, 10) adults who need fatigue/stress support and, 11) 50+ adults.

The essential daily supplements for each segment of the population is further divided into 1) a Morning formula taken with breakfast or by itself if breakfast is skipped. Breakfast is often a light meal or sometimes skipped and on those occasions, the morning formula of the present invention can safely be taken on an empty stomach. 2) a Mid-day formula taken with lunch and 3) an Evening formula taken with an evening snack. An additional fourth formula is added for those with special needs. The partitioning of the daily requirements of the essential micronutrients into these formulas for ingestion at specific times of the day along with the meals consumed during those times of the day is based on scientifically proven requirements and the benefits imparted by certain essential nutrients if taken during those times of the day. For example, certain micronutrients such as CoQ-10, vitamin B12, iron, resveratol and L-carnitine have proven to be beneficial for energy production and therefore taking them during the first half of the day along with breakfast and lunch would help keep a person more alert to do the day's work. Other micronutrients such as Calcium, Magnesium, B6, B3, Chamomile, Hops and Motherwort are known to have a relaxing effect on the body and beneficial in promoting sleep. Therefore, these nutrients are best taken in the evening.

A related consideration in grouping the vitamins, minerals and other micronutrients into the respective formulas is the type of food consumed during the respective meals- the meal content. It is well known that most essential vitamins and minerals are best absorbed when taken with food. However, the multivitamin and mineral supplements available in the market today have not taken into consideration the positive and negative interactions of the ingredients in the food with the multivitamins and minerals taken along with the food. Statistics show that most milk, coffee and tea are consumed at breakfast. Both calcium from milk and polyphenols and tannins from coffee and tea greatly inhibit iron absorption (together as much as 80%). Therefore, iron containing supplements should be taken with a meal other than breakfast. Statistics also show that most dinner meals contain meat or fish which are rich in iron. Therefore, it would be beneficial to take calcium separate from that meal. Not many people drink coffee/tea with lunch, so it would be the preferred time to take an iron-containing supplement. Another consideration is that breakfast is quite frequently skipped or is a very light meal for many people, so it would be best to include fat-soluble vitamins and nutrients (A, E, K, D, carotenoids, Co-Q10) with a meal other than breakfast, because it would be more likely that this meal would contain fat necessary for their efficient absorption

In the preferred embodiment of the invention, emphasis is placed on the interaction between common ingredients in the meal and the supplement formulas taken with that meal. The Morning formula that is taken with breakfast does not contain iron or calcium to avoid strong negative effects that coffee has on their absorption. The Mid-day formula taken with lunch contains fat-soluble vitamins since this meal is very likely to contain some fat. It also includes iron since this meal is not likely to contain a lot of calcium. Calcium is included in the Evening formula intended to be taken with an evening snack since dinner often includes meat or fish. Nutrients in the Evening formula are well absorbed without much food, so a light snack such as a cracker or a piece of fruit is sufficient. This Evening formula can also be taken with dinner if the meal does not contain much iron-rich foods. If the person has a special dietary regimen, then he/she can adjust with which meal to take each individual formula to optimize its absorption.

The other significant and distinguishing feature of the micronutrient supplements of the present invention is the grouping of the micronutrients into each formula based on their synergism and/or antagonism for each other. Antagonism between absorption of certain vitamins, minerals and other micronutrients taken at the same time has been well documented in scientific literature. Some examples are the interactions between absorption of Fe and Zn, Mg and Mn, Ca and Fe, Ca and Zn, Cu and Zn, and others. Also, synergism between absorption of other micronutrients is also well known and documented. Some examples are the synergism between absorption of Fe and C taken at the same meal, Ca and vitamin D, vitamin C and bioflavonoids, quercetin and resveratrol, and others. In the present invention, the vitamins and minerals that complement and enhance absorption of each other are combined in the same formula whereas the vitamins and minerals that antagonize and fight each other for absorption are separated and placed in different formulas. Sometimes it is not possible to avoid all the negative interactions. For practical purposes three to four formulas have been created in the present invention. Other formulas are also envisioned in the future taking into consideration the strongest synergism and antagonism as criteria for dividing the micronutrients.

In the preferred embodiment, all the interactions discussed thus far are accounted for in the formulas. There exist conflicting data regarding interaction between some nutrients. Also, interactions between some nutrients have not been sufficiently studied. Thus scientific research in this realm will be monitored and formulas updated as more information about synergism and antagonism between the micronutrients becomes available. In general, formulation of the micronutrient supplements based on these principles of synergism and antagonism between the nutrients assures the optimal absorption and utilization of the nutrients from each formula.

The micronutrient supplements of the present invention have placed a great deal of emphasis on the use of the most bio-available form of the vitamins and minerals. Most multivitamin and mineral supplements available in the market often include the cheapest, most concentrated, or most easily available sources without regard to their absorption and bioavailability. For example, selenium is available as sodium selenate and sodium selenite, which are rather inexpensive inorganic forms of selenium, they are widely used in common drugstore multivitamin supplements, but have only about 50% absorption and retention rate. On the other hand, selenomethionine which is used in the micronutrient supplement formulations of the current invention is an organic form of selenium that occurs naturally in foods and is much better absorbed by the body (about 90% is absorbed), so it should be the preferred form of selenium to be used in supplements. Another example is vitamin E which is available both in its natural form as d-alpha-tocopherol and synthetic form as dl-alpha-tocopherol. The synthetic form is less bio-available and about half as potent as the natural form.

Special emphasis has been placed on the medium of delivery of the micronutrients used in the supplement formulas of the present invention. In general, the formulas that contain fat soluble vitamins include fat in the form of Omega-3 fish oil, flax seed oil and other beneficial oils that facilitate absorption of the nutrients. Some formulas of the present invention may be in liquid form to further hasten absorption of the nutrients. Some formulas can be in sublingual form to facilitate the absorption of certain nutrients, especially B vitamins. Certain condition-specific formulas include nutrients that are best taken on an empty stomach and include special formulas that need to be taken between meals.

Most supplements that list herbs as ingredients do not use standardized extracts of the herbs. If standardized extracts of herbs are not used, the potency and efficacy of such herbal preparations can be minimal and inconsistent. For example, standardized extract of bilberry contains 25% (or other percent) of proanthocyanins and anthocyanins; standardized extract of chamomile contains 10% (or other percent) of alpha-bisabolol and should be used in place of non-standardized forms of these herbs. In the preferred embodiment of the present invention, all formulas contain vitamin and mineral forms chosen based on their bio-availability and suitability for use in a supplement and herbs are used as standardized extracts.

Human nutritional requirements vary significantly with age. Therefore, the preferred embodiments of the present invention have special formulas for toddlers (1-3), children (4-8), juniors (9-14), adults, and 50+ adults. Nutritional requirements also vary from male to female, although this variation is not significant for most nutrients, with the exception of iron, magnesium, vitamin K and some other nutrients. In the preferred embodiment, the formulas are age-specific and are based on Recommended Dietary Allowances (RDA) and Adequate Intake (AI) norms for nutrients. For nutrients that do not have established RDA or AI norms, amounts are based on the amount that can be obtained from a healthy diet rich in fruits, vegetables and nutrient-dense foods. Age and gender- specific formulas have been created based on these criteria.

In the preferred embodiment, amounts of the micronutrients are chosen to be reasonable and comparable to the amounts that individuals consume from normal balanced diets. The Recommended Dietary Allowances (RDA) and Adequate Intake (AI) norms are used for this determination. It should be noted that these standards are being updated regularly and are significantly different for several nutrients from the widely used Daily Values (DV) that were established in 1968. For example, DV for vitamin A is 5000 IU, while RDA for men is 2700 IU and 2100 IU for women; DV for zinc is 15 mg, while RDA for men is 11 mg and 8 mg for women; DV for copper is 2 mg, while RDA for men and women is 0.9 mg. These are examples of elements that can accumulate in the body, so excessive intake over a long period of time can be detrimental. Another consideration for choosing the amounts of micronutrients in the formulas of the present invention is the serving size of the formula in relation to the amounts of the nutrients consumed from the diet. Some supplements available in the market include mega-doses of vitamins. It is not known if such quantities are safe and/or beneficial over long term use. Amounts of super-foods and herbs in the preferred embodiment of the present invention are comparable to normal every-day consumption. Increased doses might be used in condition-specific formulas.

The embodiments of the present invention use special formulas for individuals with special requirements in the population as for instance, athletes, pregnant and lactating women, individuals under stress, vegetarians and others. Formulas have also been created that are specific to health conditions such as diabetes, osteoporosis, and anemia to name a few. Special formulas are also created for cardio vascular health, cholesterol control etc. In these formulas, some nutrients might be eliminated and additional nutrients incorporated. It might be necessary to increase the serving size, such as for example from 1 tablet to 2 tablets or from 20 ml to 30 ml. Also, it might be beneficial to introduce the fourth formula to help synergism between the components. These special essential formulas are beneficial because they are balanced and would insure extra essential nutrition without making the amount of certain nutrients excessive or causing antagonistic actions between nutrients. Many condition-specific formulas in the market besides condition-specific micronutrients also include essential vitamins and minerals in the amounts close to RDA or even more. Thus when those formulas are taken together with a daily multivitamin and mineral supplement, it can result in consumption of excessive doses of some vitamins and minerals. In the case of fat-soluble vitamins and minerals it can lead to excessive accumulation of these nutrients in the body, which can be detrimental. The special requirements and condition-specific essential formulas of the present invention include essential vitamins and minerals in the appropriate amounts plus additional nutrients beneficial for these conditions.

In summary, the proposed supplement formulations in the preferred embodiments of the present invention consist of three (morning, mid-day, evening) or four (morning, lunch, dinner, evening) daily formulas that have been formulated according to a 7-level optimization protocol. It is also possible to use the same principles and segregate the nutrients further into five or more other formulas when even more optimized absorption is required. Under certain constraints it might be necessary to have only two formulas and in that case, they will be grouped according to the strongest synergism and antagonism between the components and their absorption. In the preferred embodiment, the daily formulas contain at least five vitamins and at least three essential minerals and additional components such as phytochemicals, plant-derived compounds or their analogs, essential fatty acids, enzymes, herbal extracts and others. Also, in the preferred embodiment the formulas are distinctly different; out of the three or four daily formulas at least two formulas have no more than three vitamins and two minerals included that are the same.

Referring now to the Tables which describe each of the micronutrient supplement formulations based on the 7-level optimization protocol, Table 1 shows the list of micronutrients, both vitamins and minerals that have been used in the formulation of the micronutrient supplements of the present invention. In all formulations of the present invention, the most bio-available form of the vitamin or mineral is used. This list is not comprehensive since for some vitamins and minerals there are several forms with similar bio-availability or insufficient scientific data to clearly define which form is the best. Also, some of the highly bio-available forms contain only small percentage of the desired vitamins and minerals and might not be practical for supplementation purposes. As more scientific information becomes available, other more bio-available compounds of both the vitamins and minerals may be used in the preferred embodiments of the invention.

TABLE 1

| Vitamins | | Minerals | |
|---|---|---|---|
| Name | Form | Name | Form |
| A | Beta-carotine and retinyl | Calcium (Ca) | Calcium citrate, calcium malate |
| | palmitate | Chromium (Cr) | Chromium nicotinate |
| C | Ascorbic acid | Copper (Cu) | Copper glycinate chelate, copper |
| D | D3, cholecalciferol | | gluconate |
| E | D-alpha tocopheryl succinate | Iodine (Io) | Potassium iodide |
| K | Menaquinone | Iron (Fe) | Iron Glycinate Chelate, Ferrochel ® |
| B1 | Thiamin hydrochloride | Magnesium (Mg) | Magnesium aspartate, magnesium |
| B2 | Riboflavin-5-phosphate | | malate |
| B3 | Nicotinamide | Manganese (Mn) | Manganese citrate |
| B5 | Calcium pantothenate | Molybdenum (Mo) | Sodium molybdate, molybdenum |
| B6 | Pyridoxal-5-phosphate | | glycinate chelate |
| B7 | Biotin | Selenium (Se) | L-selenomethionine |
| B9 | Folic acid | Zinc (Zn) | Zinc gluconate, zinc glycinate |
| B12 | Cyanocobalamin | | chelate |
| Choline | Choline bitartrate | Potassium(K) | Potassium aspartate |

Table 2 gives the micronutrient supplement formulas suitable for adults. The Morning formula taken with breakfast contains zinc, manganese, B vitamins, vitamin C with citrus bioflavonoids complex, phytochemicals and other elements. The Mid-day formula contains iron, copper, fat soluble vitamins, carotenoids, CoQ-10, omega-3 oils that contain the essential fatty acids, DHA and EPA (in either liquid or dry fish oil form), selenium, and several other vitamins. For vegetarians, the essential fatty acids, DHA and EPA can be derived from algae, flax seed oil and other vegetarian oil sources. If this formula is provided in the liquid form, it would be beneficial to add ALA-containing oils such as flax seed oil. Morning and mid-day formulas include vitamin B12, CoQ-10, iron and other elements important in the energy production. The Evening formula contains calcium with D3, magnesium, potassium, B vitamins and herbs. It is beneficial to take calcium and magnesium in the evening, because of the relaxing nature of these minerals. B vitamins included in this formula are the ones that can help with restful sleep since B6 is involved in serotonin production, B5 is involved in the synthesis of melatonin, B3 has been shown to increase REM sleep. The amounts of most or all of the nutrients in these formulas can be reduced in half or more if necessary for individuals who follow a healthy diet and would like lighter micronutrient support. This can be achieved by making the serving size if in tablet form to two or three tablets/capsules/soft-gels so that one tablet, which corresponds to half or a third of serving size, can be ingested. Serving size of liquid formulas can similarly be adjusted. Formulas that are in liquid form may be ingested in half serving size or smaller sizes as needed.

Of the essential minerals, sodium, chloride, sulfur and phosphorus are not included in these formulas because they are generally consumed in adequate amounts from the diet. Potassium is included in a small amount because of its synergistic action with magnesium. Fluoride is not included, because most water is fluoridated.

TABLE 2
Essential Daily Micronutrient Supplement for Adults.

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 1.5 | mg | A (50% beta- | 2500 | IU | B3 | 20 | mg |
| B2 | 1.7 | mg | carotine, 50% | | | B5 | 10 | mg |
| B7 | 100 | mcg | retinyl | | | B6 | 2 | mg |
| Choline | 150 | mg | palmitate) | | | Ca | 500 | mg |
| Myo-inositol | 50 | mg | Carotenoids: | | | D3 | 200 | IU |
| Io | 150 | mcg | Lutein | 5 | mg | Mg | 200 | mg |
| Zn | 12 | mg | Lycopene | 5 | mg | Potassium | 120 | mg |
| Cr | 60 | mcg | Zeaxanthin | 250 | mcg | Mo | 60 | mcg |
| Mn | 2.0 | mg | Astaxanthin | 1 | mg | Chamomile | 50 | mg |
| Trace Minerals | 100 | mg | D3 | 400 | IU | Hops | 50 | mg |
| C | 50 | mg | E | 45 | IU | Lemon Balm | 50 | mg |
| Citrus | 100 | mg | C | 50 | mg | | | |
| bioflavonoids | | | K2 | 30 | mcg | | | |
| Rutin | 40 | mg | B9 | 400 | mcg | | | |
| Quercetin | 30 | mg | B12 | 12 | mcg | | | |
| Red Wine Extract | 60 | mg | Fe | 7 | mg | | | |
| Hawthorn | 120 | mg | Cu | 0.7 | mg | | | |
| Bilberry | 100 | mg | Se | 70 | mcg | | | |
| Pomegranate | 80 | mg | DHA | 300 | mg | | | |
| Curcumin | 40 | mg | EPA | 600 | mg | | | |
| Cinnamon | 30 | mg | CoQ-10 | 15 | mg | | | |

The choice of three main formulas allows for the elimination of some of the strongest known interactions, such as iron with calcium, calcium with zinc, zinc with iron and copper, magnesium with manganese and calcium. Although having more than three formulas is beneficial to further increase the synergism between the components, compliance in taking more than three formulas a day may pose a problem. Having nutrients divided into more than three formulas is especially beneficial in condition-specific formulas. It is to be noted that as more scientific data becomes available on other nutrients and their benefits, the formulas will be updated to include those other nutrients.

When creating formulas for other age groups, quantities in the essential formula in Table 2 will be adjusted according to RDAs for essential toddlers' (1-3), children's (4-8), juniors (9-14) and adults 50+ formulas. Segregation of nutrients would be the same as in adult's essential formula since those are balanced according to nutrient interaction and meal content.

Table 3 presents essential daily micronutrient supplements for an adult female and Table 4 presents essential daily micronutrient supplements for an adult male. The formulas are essentially similar; the main differences are in the amount of Iron, Zinc, Vitamin K, Chromium, Vitamin A and Manganese. Table 5 shows an example of the essential daily micronutrient supplements for children ages 4-8.

TABLE 3
Essential Daily Micronutrient Supplement for a Female

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 1.5 | mg | A (50% beta- | 2100 | IU | B3 | 20 | mg |
| B2 | 1.5 | mg | carotine, 50% | | | B5 | 10 | mg |
| B7 | 100 | mcg | retinyl | | | B6 | 2 | mg |
| Choline | 150 | mg | palmitate) | | | Ca | 500 | mg |
| Myo-inositol | 50 | mg | Carotenoids: | | | D3 | 200 | IU |
| Io | 150 | mcg | Lutein | 5 | mg | Mg | 200 | mg |
| Zn | 10 | mg | Lycopene | 5 | mg | Potassium | 120 | mg |
| Cr | 50 | mcg | Zeaxanthin | 250 | mcg | Mo | 60 | mcg |
| Mn | 1.8 | mg | Astaxanthin | 1 | mg | Chamomile | 50 | mg |
| Trace Minerals | 100 | mg | D3 | 400 | IU | Hops | 50 | mg |
| C | 50 | mg | E | 45 | IU | Lemon Balm | 50 | mg |
| Citrus | 100 | mg | C | 50 | mg | | | |
| bioflavonoids | | | K2 | 30 | mcg | | | |
| Rutin | 40 | mg | B9 | 400 | mcg | | | |
| Quercetin | 30 | mg | B12 | 12 | mcg | | | |
| Red Wine Extract | 60 | mg | Fe | 18 | mg | | | |
| Hawthorn | 120 | mg | Cu | 0.7 | mg | | | |
| Bilberry | 100 | mg | Se | 70 | mcg | | | |
| Pomegranate | 80 | mg | DHA | 300 | mg | | | |
| Curcumin | 40 | mg | EPA | 600 | mg | | | |
| Cinnamon | 30 | mg | CoQ-10 | 15 | mg | | | |

TABLE 4
Essential Daily Micronutrient Supplement for Adult Males.

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 1.5 | mg | A (50% beta- | 2500 | IU | B3 | 20 | mg |
| B2 | 1.7 | mg | carotine, 50% | | | B5 | 10 | mg |
| B7 | 100 | mcg | retinyl | | | B6 | 2 | mg |
| Choline | 150 | mg | palmitate) | | | Ca | 500 | mg |
| Myo-inositol | 50 | mg | Carotenoids: | | | D3 | 200 | IU |
| Io | 150 | mcg | Lutein | 5 | mg | Mg | 250 | mg |
| Zn | 12 | mg | Lycopene | 5 | mg | Potassium | 120 | mg |
| Cr | 70 | mcg | Zeaxanthin | 250 | mcg | Mo | 60 | mcg |
| Mn | 2.3 | mg | Astaxanthin | 1 | mg | Chamomile | 50 | mg |
| Trace Minerals | 100 | mg | D3 | 400 | IU | Hops | 50 | mg |
| C | 50 | mg | E | 45 | IU | Lemon Balm | 50 | mg |
| Citrus | 100 | mg | C | 50 | mg | | | |
| bioflavonoids | | | K2 | 40 | mcg | | | |
| Rutin | 40 | mg | B9 | 400 | mcg | | | |
| Quercetin | 30 | mg | B12 | 12 | mcg | | | |
| Red Wine Extract | 60 | mg | Fe | 7 | mg | | | |
| Hawthorn | 120 | mg | Cu | 0.7 | mg | | | |
| Bilberry | 100 | mg | Se | 70 | mcg | | | |
| Pomegranate | 80 | mg | DHA | 300 | mg | | | |
| Curcumin | 40 | mg | EPA | 600 | mg | | | |
| Cinnamon | 30 | mg | CoQ-10 | 15 | mg | | | |

TABLE 5
Essential Daily Micronutrient Supplement for Children 4-8.

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 0.6 | mg | A (50% beta- | 1333 | IU | B3 | 8 | mg |
| B2 | 0.6 | mg | carotine, 50% | | | B5 | 3 | mg |
| B7 | 20 | mcg | retinyl | | | B6 | 0.6 | mg |
| Choline | 150 | mg | palmitate) | | | Ca | 500 | mg |
| Myo-inositol | 20 | mg | Carotenoids: | | | D3 | 200 | IU |
| Io | 90 | mcg | Lutein | 2.5 | mg | Mg | 100 | mg |
| Zn | 5 | mg | Lycopene | 2.5 | mg | Potassium | 80 | mg |
| Cr | 15 | mcg | Zeaxanthin | 125 | mcg | Mo | 22 | mcg |
| Mn | 1.5 | mg | Astaxanthin | 500 | mcg | Chamomile | 50 | mg |
| TraceMinerals | 50 | mg | D3 | 400 | IU | Lemon Balm | 50 | mg |
| C | 25 | mg | E | 12 | IU | | | |
| Citrus | 60 | mg | C | 25 | mg | | | |
| bioflavonoids | | | K2 | 30 | mcg | | | |
| Rutin | 20 | mg | B9 | 200 | mcg | | | |
| Quercetin | 20 | mg | B12 | 1.2 | mcg | | | |
| Bilberry | 120 | mg | Fe | 8 | mg | | | |
| Pomegranate | 100 | mg | Cu | 0.44 | mg | | | |
| Curcumin | 40 | mg | Se | 30 | mcg | | | |
| Cinnamon | 40 | mg | DHA | 600 | mg | | | |
| | | | EPA | 200 | mg | | | |

Some embodiments of the present invention comprise special supplements for other segments of the population such as pregnant and lactating women as shown in Table 6 and for athletes, vegetarians and others (not shown).

TABLE 6
Essential Daily Micronutrient Supplement for Pregnant Women

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 1.5 | mg | A (50% beta- | 2500 | IU | B3 | 20 | mg |
| B2 | 1.7 | mg | carotine, 50% | | | B5 | 10 | mg |
| B7 | 100 | mcg | retinyl | | | B6 | 2 | mg |
| Choline | 200 | mg | palmitate) | | | Ca | 500 | mg |
| Myo-inositol | 50 | mg | Carotenoids: | | | D3 | 200 | IU |
| Io | 220 | mcg | Lutein | 5 | mg | Mg | 200 | mg |
| Zn | 15 | mg | Lycopene | 5 | mg | Potassium | 120 | mg |
| Cr | 80 | mcg | Zeaxanthin | 250 | mcg | Mo | 60 | mcg |
| Mn | 2.3 | mg | Astaxanthin | 1 | mg | Chamomile | 50 | mg |
| Trace Minerals | 100 | mg | D3 | 400 | IU | Hops | 50 | mg |
| C | 50 | mg | E | 45 | IU | Lemon Balm | 50 | mg |
| Citrus | 100 | mg | C | 50 | mg | | | |
| bioflavonoids | | | K2 | 90 | mcg | | | |
| Rutin | 40 | mg | B9 | 600 | mcg | | | |
| Quercetin | 30 | mg | B12 | 12 | mcg | | | |
| Hawthorn | 120 | mg | Fe | 22 | mg | | | |
| Bilberry | 100 | mg | Cu | 1 | mg | | | |
| Raspberry Leaf | 100 | mg | Se | 70 | mcg | | | |
| Pomegranate | 80 | mg | DHA | 700 | mg | | | |
| Curcumin | 40 | mg | EPA | 100 | mg | | | |
| Cinnamon | 30 | mg | CoQ-10 | 20 | mg | | | |

The preferred embodiments of the present invention also have condition -specific micronutrient supplements and related formulas suitable for individuals with certain health conditions. These formulas are based on the 7-level protocol that includes extra nutrients that are beneficial for each condition. Table 7 shows an example of an essential micronutrient supplement for cardio/cholesterol support with added nutrients such as resveratol, grape seed extract, L-carnitine, garlic extract, extra CoQ-10 and motherwort.

TABLE 7
Essential Daily Micronutrient Supplement for Adults with Cardio/Cholesterol Support

| Morning Formula | | | Mid-day Formula | | | Evening Formula | | |
|---|---|---|---|---|---|---|---|---|
| (taken with breakfast) | | | (taken with lunch) | | | (taken with evening snack) | | |
| Micronutrient | Amount | | Micronutrient | Amount | | Micronutrient | Amount | |
| B1 | 1.5 | mg | A (50% beta- | 2500 | IU | B3 | 20 | mg |
| B2 | 1.7 | mg | carotine, 50% | | | B5 | 10 | mg |
| B7 | 100 | mcg | retinyl | | | B6 | 2 | mg |
| Choline | 150 | mg | palmitate) | | | Ca | 500 | mg |
| Myo-inositol | 50 | mg | Carotenoids: | | | D3 | 200 | IU |
| Io | 150 | mcg | Lutein | 5 | mg | Mg | 250 | mg |
| Zn | 12 | mg | Lycopene | 5 | mg | Potassium | 140 | mg |
| Cr | 80 | mcg | Zeaxanthin | 250 | mcg | Mo | 60 | mcg |
| Mn | 2.0 | mg | Astaxanthin | 1 | mg | Motherwort | 80 | mg |
| Trace Minerals | 100 | mg | D3 | 400 | IU | Chamomile | 50 | mg |
| C | 50 | mg | E | 60 | IU | Hops | 50 | mg |
| Citrus | 100 | mg | C | 50 | mg | Lemon Balm | 50 | mg |
| bioflavonoids | | | K2 | 80 | mcg | | | |
| Rutin | 40 | mg | B9 | 400 | mcg | | | |
| Quercetin | 30 | mg | B12 | 12 | mcg | | | |
| T-resveratrol | 5 | mg | Fe | 7 | mg | | | |
| Red Wine Extract | 100 | mg | Cu | 0.7 | mg | | | |
| Grape seed extract | 100 | mg | Se | 70 | mcg | | | |
| L-carnitine | 200 | mg | DHA | 400 | mg | | | |
| Hawthorn | 200 | mg | EPA | 600 | mg | | | |
| Bilberry | 100 | mg | CoQ-10 | 40 | mg | | | |
| Pomegranate | 80 | mg | | | | | | |
| Curcumin | 40 | mg | | | | | | |
| Cinnamon | 30 | mg | | | | | | |

Since some of these added nutrients have antagonistic actions with components of the original formula, it was found to be more beneficial to divide this supplement into four formulas as shown in Table 8. This allows for the separation of minerals from the polyphenols, especially the ones from the grape seed extract that are found to be very beneficial for cardio protection, but also known to significantly hinder mineral absorption.

TABLE 8
Essential Daily Micronutrient Supplement with Cardio/Cholesterol Support

| Morning Formula | | | Mid-Day Formula | | | Evening Formula 1 | | | Evening Formula 2 | | |
|---|---|---|---|---|---|---|---|---|---|---|---|
| (1 hr before or after breakfast) | | | (with lunch) | | | (with dinner) | | | (with evening snack) | | |
| Micronutrient | Amount | | Micronutrient | Amount | | Micronutrient | Amount | | Micronutrient | Amount | |
| C | 50 | mg | A (50% beta- | 2500 | IU | B1 | 1.5 | mg | B3 | 20 | mg |
| Citrus | 100 | mg | carotine, 50% | | | B2 | 1.7 | mg | B5 | 10 | mg |
| bioflavonoids | | | retinyl palmitate) | | | B7 | 100 | mcg | B6 | 2 | mg |
| Rutin | 40 | mg | Carotenoids: | | | Choline | 200 | mg | Ca | 500 | mg |
| Quercetin | 30 | mg | Lutein | 5 | mg | Myo-inositol | 50 | mg | D3 | 200 | IU |
| T-resveratrol | 5 | mg | Lycopene | 5 | mg | Io | 150 | mcg | Mg | 250 | mg |
| Red Wine | 100 | mg | Zeaxanthin | 250 | mcg | Zn | 12 | mg | Potassium | 140 | mg |
| Exract | | | Astaxanthin | 1 | mg | Cr | 80 | mcg | Mo | 60 | mcg |
| Grape seed | 100 | mg | D3 | 400 | IU | Mn | 2.0 | mg | Motherwort | 80 | mg |
| extract | | | E | 60 | IU | Trace Minerals | 200 | mg | Chamomile | 50 | mg |
| L-carnitine | 200 | mg | C | 50 | mg | Aged garlic | 200 | mg | Hops | 50 | mg |
| Hawthorn | 200 | mg | K2 | 80 | mcg | extract | | | Lemon Balm | 50 | mg |
| Bilberry | 100 | mg | B9 | 400 | mcg | | | | | | |
| Pomegranate | 80 | mg | B12 | 12 | mcg | | | | | | |
| Curcumin | 40 | mg | Fe | 7 | mg | | | | | | |
| Cinnamon | 30 | mg | Cu | 0.7 | mg | | | | | | |
| | | | Se | 70 | mcg | | | | | | |
| | | | DHA | 400 | mg | | | | | | |
| | | | EPA | 600 | mg | | | | | | |
| | | | CoQ-10 | 40 | mg | | | | | | |

Table 9 shows a special supplement for iron deficiency in adults. This supplement is divided into four formulas to optimize absorption. Using three formulas would create a number of undesirable interactions. The morning formula already contains zinc, therefore, addition of iron would cause antagonistic action between the nutrients. Also, polyphenols from coffee/tea often consumed in the morning would hinder iron absorption. Mid-day formula already contains iron and for best absorption larger doses of iron should be broken into several smaller ones. Evening formula contains calcium that hinders iron absorption. Thus the best time to include extra iron is to increase amount of iron in the mid-day formula and introduce the fourth formula with iron taken with dinner.

TABLE 9
Essential Daily Micronutrient Supplement for Adults with Iron-deficiency.

| Morning Formula | | | Mid-day Formula | | | Evening Formula 1 | | Evening Formula 2 | | |
|---|---|---|---|---|---|---|---|---|---|---|
| (1 hr before or after breakfast) | | | (with lunch) | | | (with dinner) | | (with evening snack) | | |
| Micronutrient | Amount | | Micronutrient | Amount | | Micronutrient | Amount | Micronutrient | Amount | |
| B1 | 1.5 | mg | A (50% beta- | 2500 | IU | C | 80 mg | B3 | 20 | mg |
| B2 | 1.7 | mg | carotine, 50% | | | Fe | 15 mg | B5 | 10 | mg |
| B7 | 100 | mcg | retinyl palmitate) | | | Liver | 900 mg | B6 | 2 | mg |
| Choline | 150 | mg | Carotenoids: | | | fractions | | Ca | 500 | mg |
| Myo-inositol | 50 | mg | Lutein | 5 | mg | | | D3 | 200 | IU |
| Io | 150 | mcg | Lycopene | 5 | mg | | | Mg | 200 | mg |
| Zn | 12 | mg | Zeaxanthin | 250 | mcg | | | Potassium | 140 | mg |
| Cr | 60 | mcg | Astaxanthin | 1 | mg | | | Mo | 60 | mcg |
| Mn | 2.0 | mg | D3 | 400 | IU | | | Chamomile | 50 | mg |
| Trace Minerals | 100 | mg | E | 60 | IU | | | Hops | 50 | mg |
| C | 50 | mg | C | 80 | mg | | | Lemon Balm | 50 | mg |
| Citrus | 100 | mg | K2 | 80 | mcg | | | | | |
| bioflavonoids | | | Fe | 15 | mg | | | | | |
| Rutin | 40 | mg | Cu | 0.7 | mg | | | | | |
| Quercetin | 30 | mg | Se | 70 | mcg | | | | | |
| Red Wine | 60 | mg | DHA | 200 | mg | | | | | |
| Extract | | | EPA | 400 | mg | | | | | |
| Hawthorn | 120 | mg | CoQ-10 | 30 | mg | | | | | |
| Bilberry | 100 | mg | | | | | | | | |
| Pomegranate | 80 | mg | | | | | | | | |
| Curcumin | 40 | mg | | | | | | | | |
| Cinnamon | 30 | mg | | | | | | | | |

Table 10 shows an example of a special supplement according to one embodiment of the present invention. This supplement and its associated four formulas are specifically created for individuals under stress and/or who exhibit fatigue symptoms. It is also possible to create a three formula supplement for these conditions. However, there will be more undesirable interactions between the components.

TABLE 10
Essential Daily Micronutrient Supplement for Adults with Fatigue/Stress support.

| Morning Formula | | | Mid-day Formula | | | Evening Formula 1 | | | Evening Formula 2 | | |
|---|---|---|---|---|---|---|---|---|---|---|---|
| (1 hr before or after breakfast) | | | (with lunch) | | | (with dinner) | | | (with evening snack) | | |
| Micronutrient | Amount | | Micronutrient | Amount | | Micronutrient | Amount | | Micronutrient | Amount | |
| B1 | 1.5 | mg | A (50% beta- | 2500 | IU | B1 | 1.5 | mg | B3 | 20 | mg |
| B2 | 1.7 | mg | carotine, 50% | | | B2 | 1.7 | mg | B5 | 10 | mg |
| B3 | 20 | mg | retinyl palmitate) | | | B7 | 100 | mcg | B6 | 2 | mg |
| B5 | 10 | mg | Carotenoids: | | | Choline | 200 | mg | Ca | 500 | mg |
| B6 | 2 | mg | Lutein | 5 | mg | Myo-inositol | 50 | mg | D3 | 200 | IU |
| B7 | 100 | mcg | Lycopene | 5 | mg | Io | 150 | mcg | Mg | 250 | mg |
| B9 | 400 | mcg | Zeaxanthin | 250 | mcg | Zn | 12 | mg | Potassium | 140 | mg |
| B12 | 18 | mcg | Astaxanthin | 1 | mg | Cr | 80 | mcg | Mo | 60 | mcg |
| Myo-inositol | 50 | mg | D3 | 400 | IU | Mn | 2.0 | mg | Motherwort | 80 | mg |
| C | 50 | mg | E | 60 | IU | Trace | 200 | mg | Chamomile | 50 | mg |
| Citrus | 100 | mg | C | 50 | mg | Minerals | | | Hops | 50 | mg |
| bioflavonoids | | | K2 | 80 | mcg | Curcumin | 40 | mg | Lemon Balm | 50 | mg |
| Rutin | 40 | mg | Fe | 15 | mg | Cinnamon | 30 | mg | | | |
| Quercetin | 30 | mg | Cu | 0.7 | mg | Ashwagandha | 250 | mg | | | |
| Mg | 100 | mg | Se | 70 | mcg | | | | | | |
| Potassium | 80 | mg | DHA | 400 | mg | | | | | | |
| T-resveratrol | 15 | mg | EPA | 600 | mg | | | | | | |
| Red Wine | 100 | mg | CoQ-10 | 40 | mg | | | | | | |
| Extract | | | | | | | | | | | |
| Grape seed | 100 | mg | | | | | | | | | |
| extract | | | | | | | | | | | |
| L-carnitine | 200 | mg | | | | | | | | | |
| Holy Basil | 100 | mg | | | | | | | | | |
| Hawthorn | 200 | mg | | | | | | | | | |
| Pomegranate | 100 | mg | | | | | | | | | |

The formulas discussed above can be presented in any suitable delivery medium known in the art such as tablets, capsules, liquid-filled capsules, liquids, liquid single serving containers (pouches, shots, etc.), effervescent tablets or powders, sublingual tablets, gummy-type supplements, nutritional bars, meal-replacement products and many others. These formulas can also be part of modified functional foods. When necessary, binders, lubricants, absorbents, preservatives and other ingredients necessary to incorporate and preserve micronutrients can be added. Whenever possible, the emphasis should be on all-natural form with minimal use of artificial additives.

Formulas presented here are illustrations of using the 7-level optimization protocol for creation of a daily essential multi-micronutrient supplement. Exact nutrients and amounts of nutrients in the formulas can be modified to serve particular needs. Other formulas can be created based on the same principles. It is also possible to create a formulation where the formulas are distributed between the two consecutive days. It is also possible to separate additional nutrients used in condition specific formulas into add-on formulas that will be taken either with one of the three essential formulas or between them. It is possible to create formulas with only vitamins and minerals without addition of any other nutrients. Also, the first three levels of our 7-level optimization protocol can be used by themselves or as subset of another optimization protocol. All such formulas will have high degree of synergism between the components and optimized absorption and are part of the present invention.

The foregoing description of the present invention through its various embodiments should not be construed to limit the scope of the invention. It should be understood and obvious to one skilled in the art that alternatives, modifications, and variations of the embodiments of the present invention may be construed as being within the spirit and scope of the invention as set forth in the appended claims.

What is claimed is:

1. Dietary micronutrient supplement formulations for daily administration to humans comprising:
micronutrients such as vitamins, minerals, phytochemicals, fish and plant oils, amino acids, enzymes, herb and fruit extracts and other natural compounds;
said micronutrients incorporated into supplements for intake by different segments of the population based on age, gender, special requirements and health conditions;
said supplements comprising formulas to be administered along with meals, without meals or in between meals at different times of the day;
said micronutrients grouped into said formulas based on their interactions with the ingredients in the foods consumed during the meals at different times of the day;
said micronutrients grouped into said formulas based on their synergism and antagonism towards each other; and
said micronutrients grouped into said formulas based on their bioavailability.

2. The dietary micronutrient supplement formulations of claim 1 wherein the micronutrient vitamins are selected from a group comprising, vitamins, A, C, D, E, K, B1, B2, B3, B5, B6, B7, B9, B12, pantothenic acid, choline, and myo-inositol.

3. The dietary micronutrient supplement formulations of claim 1 wherein the micronutrient minerals are selected from a group comprising, Ca, Mg, Zn, Fe, Cu, Mn, Cr, Io, Se, Mo, potassium and other trace minerals.

4. The dietary micronutrient supplement formulation of claim 1 wherein the micronutrient phytochemicals are selected from a group comprising CoQ-10, resveratol, lutein, lycopene, astaxanthin, zeaxanthin, rutin, quercetin and citrus bioflavonoids.

5. The dietary micronutrient supplement formulations of claim 1 wherein the other natural compounds are selected from standardized extracts of herbs and fruits, amino acids, red wine extract, grape seed extract, hawthorn, motherwort, bilberry, pomegranate, curcumin, cinnamon, ashwagandha, chamomile, hops, lemon balm, aged garlic extract, 1-carnitine, liver fractions, omega 3 fish oil, flax seed oil, evening primrose oil and other natural compounds.

6. The dietary micronutrient supplement formulations of claim 1 wherein the supplement for each segment of the population based on age, gender, special requirements and health conditions may be divided into two, three, four or more formulas of the micronutrients, depending on their synergism and antagonism with each other and the level of optimized absorption of the nutrients that is required based on age, gender, special requirements and health conditions.

7. The dietary micronutrient supplement formulations of claim 1 wherein the three main formulas of the micronutrient supplements are, a morning formula, a mid-day formula and an evening formula based on the time of day and the meals consumed at that time of the day.

8. The dietary micronutrient supplement formulations of claim 1 wherein there may be four formulas of the micronutrient supplements, a morning formula, a mid-day formula, an evening formula 1 and an evening formula 2 based on the time of day and the meals consumed at that time of the day.

9. The dietary micronutrient supplement formulations of claim 1, wherein only two micronutrient formulas may be created based on special requirements.

10. The dietary micronutrient supplement formulations of claim 1 wherein the formulas are distributed between two consecutive days.

11. The dietary micronutrient supplement formulations of claim 1 wherein additional nutrients used in condition specific formulas are separated into add-on formulas that will be taken either with one of the three essential formulas or between them.

12. The dietary micronutrient supplement formulations of claim 1 wherein some formulas have only vitamins and minerals without addition of any other nutrients.

13. The dietary micronutrient supplement formulations of claim 1 wherein additional supplements and micronutrient formulas may be created based on special needs and health conditions.

14. The dietary micronutrient supplement formulations of claim 1 wherein the daily formulas contain at least five vitamins, at least three essential minerals and additional components such as phytochemicals, essential fatty acids such as DHA and EPA, enzymes, herbal and fruit extracts and other natural compounds.

15. The dietary micronutrient supplement formulations of claim 1 wherein out of the three or more daily formulas, at least two formulas have no more than three vitamins and two minerals that are the same.

16. The dietary micronutrient supplement formulations of claim 1 wherein the quantity of micronutrients grouped in the formulas are based on Recommended Dietary Allowances (RDA) and Adequate Intake (AI) requirements for each age and gender group.

17. The dietary micronutrient supplement formulations of claim 1 wherein the quantity of micronutrients grouped in the formulas are based on special needs and health conditions.

18. The dietary micronutrient supplement formulation of claim 1 wherein the formulas may be in any form such as, solid, semi-solid, powder, liquid, effervescent, rapidly dissolving in liquid, sublingual, time release, chewable, gummy, gum, lozenges, encapsulated, tablet or combinations of any of these forms.
